(19) United States
(12) Patent Application Publication
(10) Pub. No.: US 2026/0232569 A1
(43) Pub. Date: Aug. 13, 2026

(54) LIQUID EXTRACT FROM SPRIGS AND/OR TWIGS OR MORUS ALBA L. AND RELATED TOPICAL COMPOSITIONS FOR THE PREVENTION AND TREATMENT OF SKIN AGEING

(71) Applicant: Unifarco S.p.A., Santa Giustina BL (IT)

(72) Inventors: Giovanni Baratto, Santa Giustina (BL) (IT); Sara Ferrari, Santa Giustina (BL) (IT)

(73) Assignee: Unifarco S.p.A., Santa Giustina BL (IT)

(21) Appl. No.: 19/153,345

(22) PCT Filed: Feb. 5, 2024
(86) PCT No.: PCT/EP2024/052813

§ 371 Date: Aug. 4, 2025

(30) Foreign Application Priority Data
Feb. 6, 2023 (IT) 102023000001920

(51) Int. Cl. A61K 8/9789; A61K 8/23; A61K 8/34; A61K 8/60; A61K 8/64; A61Q 19/08

(52) U.S. Cl. CPC A61K 8/9789; A61K 8/23; A61K 8/345; A61K 8/347; A61K 8/602; A61K 8/64; A61Q 19/08

(57) ABSTRACT

Liquid extract comprising: A. a phytocomplex extracted from sprigs and/or twigs of Morus alba L., comprising 25. Oxyresveratrol, 26. Mulberroside A B. a solvent mixture in which said phytocomplex A is dissolved, said mixture comprising: i. 1,3-propanediol ii. Erythritol iii. Water wherein I) The mixture solvent B) is the extraction solvent II) the concentration of a. of the phytocomplex A is comprised between 0.01 and 0.1% by weight on the total weight of the liquid extract, preferably between 0.02 and 0.05% III) the concentration of b. of the phytocomplex A. is comprised between 0.007 and 0.07% by weight on the total weight of the liquid extract, preferably between 0.01 and 0.04% IV) the weight ratio (i.+ii.)/iii. in the solvent mixture B. is comprised between 2.00 and 2.50, preferably is 2.33, V) the weight ratio i./ii. in the solvent mixture B. is comprised between 7.75 and 16.50, preferably it is comprised between 10.66 and 13.00, VI) the weight summation of i.+ii.+iii.=100, This extract may be added as such in a composition for cosmetic use having global anti-age activity.

### FIELD OF INVENTION

The present invention concerns a liquid extract comprising a phytocomplex of twigs and/or twigs of Morus alba L. and topical compositions comprising this extract.

### STATE OF THE ART—BACKGROUND OF THE INVENTION

The Morus genus, which belongs to the Moraceae family, is consists of more than 150 species, and among these, the white mulberry (Morus alba L.) is a dominant species distributed throughout the temperate and subtropical region of the northern and southern hemispheres. It grows in a wide range of climatic and topographical conditions from mean sea level to altitudes up to 4,000 m. The tree is widely grows spontaneously in Asia and is cultivated in China, Korea, Japan, Pakistan, Afghanistan, the northern part of the trans-Indus territory and the north-western Himalayas. Various parts of this plant, including roots, fruits, twigs, leaves and root bark, have been used in traditional Chinese medicine for centuries. (Thomas Pannakal, S, Eilstein, J, Prasad, A, et al. Comprehensive characterization of naturally occurring antioxidants from the twigs of mulberry (Morus alba L.) using on-line high-performance liquid chromatography coupled with chemical detection and high-resolution mass spectrometry. Phytochemical Analysis. 2022; 33(1): 105-114. https://doi.org/10.1002/pca.3072) Morus alba L. twigs have been widely used to treat joint pain and numbness in oriental medicine and with potential health benefits against diabetes, stroke, cough. Several researchers have studied the twigs of Morus alba L. and reported their potent inhibitory effects on human and murine tyrosinases as well as on melanin synthesis in B-16 cell melanoma. The twigs of Morus alba L. contain a large reservoir of secondary metabolites, especially antioxidants which could be used in the cosmetic field for the development of skin care products and to counteract the onset of signs of aging such as wrinkles and discoloration.

In a study by Hwang et al. it is reported that the ethanol extract of Morus alba L. twigs contains mulberroside A, quercetin, quercetin-3-O-β-glucoside, in addition to the already known resveratrol and oxyresveratrol; furthermore, this extract shows a high antioxidant activity tested with in vitro assays such as 2,2-diphenyl-1-picrylhydrazyl (DPPH).

It is also known that an alcoholic extract of Morus alba L. twig, in particular, contains oxyresveratrol: this molecule is well known as a tyrosinase inhibitor and its potential anti-browning effect and as a whitening agent for the skin have been described, both in food and cosmetics, however its use in the food and cosmetic industry has not yet been implemented due to problems related to its low solubility in water, poor bioavailability and low stability when compared to other functional active substances. In recent years, several experiments have been described to overcome these problems, for example its encapsulation with cyclodextrins. (Likhitwitayawuid, K. Oxyresveratrol: Sources, Productions, Biological Activities, Pharmacokinetics, and Delivery Systems. Molecules 2021, 26, 4212. https://doi.org/10.3390/molecules26144212)

In the cosmetic field, there are several examples of extracts of Morus alba L. starting from leaves, roots, fruits and, in particular, almost all the extracts for cosmetic purposes currently on the market are obtained starting from an extraction in a hydroalcoholic solvent, more frequently in 70-80% ethanol followed by a purification step in a chromatographic column. The extract thus obtained is normally a colored powder which must be conveyed and adequately stabilized in a cosmetic formulation.

International patent application WO2020/201432 discloses a cosmetic composition comprising Mulberroside A and Mulberroside E as well as Mulberroside F, cis-oxyresveratrol-3′-O-glucopyranoside, trans-oxyresveratrol-3-O-b-glucopyranoside, Oxyresveratrol. These components are obtained through a hydroalcoholic extraction (70% ethanol) of twigs and/or sprigs of Morus alba L. which, after a purification step on resin, washing of the liquid extract obtained and evaporation of the ethanol solvent, leads to the production of a dry extract. To be used in a cosmetic formulation, this dry extract must be appropriately conveyed to guarantee its stability. The cosmetic composition thus obtained is indicated for depigmenting and lightening activities.

In application US2006/0216253 cosmetic products are disclosed, including extracts of Morus alba L., which have a whitening effect. According to the invention, the extraction of twigs of Morus alba L. in powder form is carried out with a solution of ethanol in water 30-100%, preferably 70%. This process provides an extract comprising oxyresveratrol at 0.5-5% and mulberroside A at 0.5-5% by weight on the total weight of the extract. It is also possible to obtain extracts enriched in Oxyresveratrol or Mulberroside A, as well as purified Oxyresveratrol and Mulberroside A by a chromatographic column purification process. The purified extracts or compounds can be added to conventional skin care cosmetics to provide depigmenting cosmetics as they act on the inhibition of tyrosinase, a key enzyme in the process of skin pigmentation. Also in this case, the dry extract obtained, yellow in colour, must be appropriately conveyed in the final cosmetic formulation to guarantee the stability and therefore the effectiveness of the active ingredients claimed in the patent application.

When performing solvent extraction from plant-based material, the solvent must meet the following criteria:
high selectivity for the target compound to be extracted
safety
ease of separation from target compounds
good workability
low boiling temperature
economic

Some of the conventional volatile organic solvents used in plant drug extraction, in order of increasing polarity, include petroleum ether, n-hexane, toluene, diethyl ether, chloroform, dichloromethane, ethyl acetate, acetone, n-butanol, ethanol and methanol. Generally, these solvents are used in large quantities, especially when maceration or percolation is used, causing an increase in extraction costs. The use of organic solvents is relatively toxic due to improper extraction procedures and the possibility of residual solvents in the extracts produced. The volatility of solvents makes them potential air pollutants that contribute to global warming. The use of alternative and green solvents is the subject of numerous scientific studies.

### SUMMARY OF THE INVENTION

The applicant has now surprisingly found that it is possible to produce a liquid extract, starting from twigs and/or sprigs of Morus alba L., without the use of organic solvents such as ethanol.

In particular, this extract can be directly used in a composition for cosmetic use, and includes a phytocomplex comprising Oxyresveratrol and Mulberroside A.

The dried and ground herbal drug is mixed with a mixture containing cosmetic ingredients which the applicant has found to be capable of extracting Oxyresveratrol and Mulberroside A from the drug. The ingredients used in the solvent mixture themselves contribute to creating an already stable and suitable vehicle for a cosmetic composition for topical use, thus avoiding the solvent removal step and the production of processing waste. An extraction process was therefore identified that produces a liquid extract comparable to a raw material of vegetal origin of “cosmetic grade” and free of preservatives.

The liquid extract thus obtained can be inserted as such in a topical composition, in particular for cosmetic use with a global anti-aging action.

The liquid extract thus obtained can be used in topical formulations for mainly cosmetic use in the prevention and treatment of physiological mechanisms linked to aging and the well-being of the skin.

The object of the present invention is therefore a liquid extract comprising:
A. a phytocomplex extracted from twigs and/or sprigs of Morus alba L., comprising:
a. Oxyresveratrol,
b. Mulberroside A
B. a solvent mixture in which the phytocomplex A. is dissolved, said mixture comprising:
i. 1,3-propanediol
ii. Erythritol
iii. Water
in which:
I) the mixture solvent B is the solvent extraction
II) the concentration of a. of the phytocomplex A is between 0.01 and 0.1%, preferably between 0.02 and 0.05%, by weight of the total weight of the liquid extract,
III) the concentration of b. of the phytocomplex A. is between 0.007 and 0.07%, preferably between 0.01 and 0.04%, by weight of the total weight of the liquid extract A.,
IV) the weight ratio (i.+ii.)/iii. in solvent B. is between 2.00 and 2.50, preferably it is 2.33,
V) the weight ratio i./ii. in solvent B. it is between 7.75 and 16.50, preferably it is between 10.66 and 13.00;
VI) the weighted summation of i.+ii. +iii. =100

### DESCRIPTION OF THE FIGURES

FIG. 1 shows the histograms relating to the drop in Oxyresveratrol of the T292, T293, T294 and T295 extracts one month after preparation and subjected to the three conditions described in example 4, table 7. The values shown in the graph refer to the drop in % compared to the initial titre of active ingredient of each prototype.

FIG. 2 shows the histograms relating to the drop in Mulberroside A of the T292, T293, T294 and T295 extracts one month after preparation and subjected to the three conditions described in example 4, table 7. The values shown in the graph refer to the drop in % compared to the initial titre of active ingredient of each prototype.

### DETAILED DESCRIPTION OF THE INVENTION

For the purposes of the present invention, the definitions “comprising” or “containing” encompass the possibility that further components are present in addition to those mentioned after this definition.

On the contrary, for the purposes of the present invention the definitions “consisting of” excludes the possibility that further components are present in addition to those listed after these definitions.

For the purposes of the present invention, an extract of Morus alba L. is defined as that obtained from twigs and/or sprigs of said plant.

For the purposes of the present invention, branch, twig, and sprig refer to the botanical definition of these parts of the plant, also named as branches forming the stem, and the secondary branch is each of the parts (axes) into which the stem or root of a cormophyte is divided; the trees branches can be distinguished in primary branches, who form the scaffolding, and the branches of second order, inserted into the first ones and on which the twigs of two or more years are implanted, which in turn carry the sprigs (i.e., the branches of the year).

For this reason and for the purposes of the present invention the above twigs and sprigs fall within the more general classification of tree stem. In view of the foregoing the sprigs and/or twigs extract may be defined also as stem extracts.

The term “active ingredient” or “active” or “functional active” can be defined as any ingredient, molecule, substance, or mixture of compounds, capable of measurably stimulating a change in one of the skin parameters being evaluating, where such changes are significantly different compared to the vehicle-only application.

The term cosmetic carrier refers to the set of excipients that contribute to the cosmetic formulation and can influence the skin permeability of the active ingredients.

However, it should be considered that even a simple “carrier”, although without active ingredients, is not a completely inert cosmetic product.

For the purposes of the present invention with the wording “propanediol”, we mean the isomer “1,3 propanediol” as listed in INCI.

The synergy between vehicle and active ingredients cannot be predicted apriori and the choice of the right vehicle guarantees/supports the effectiveness and stability of the cosmetic product, especially in the case of active ingredients of plant origin.

Most of the technical problems linked to the formulation of a cosmetic product containing active ingredients of plant origin can be overcome through a careful choice of raw materials, but it is impossible to predict the interaction between the functional active ingredients and the ingredients (emulsifiers, polymers, oils) that make up the carrier.

With the term “cosmetic composition” the applicant refers to a category of consumer products whose production and marketing is subject to specific rules. The reference legislation in the European Union, and therefore also in Italy, is Regulation (EC) no. 1223/2009 of the European Parliament and of the Council of 30 Nov. 2009 on cosmetic products. As defined in Article 2 of the Regulation, “cosmetic product” means: “any substance or mixture intended to be applied to the external surfaces of the human body (epidermis, hair and hair system, nails, lips, external genital organs) or on the teeth and on the mucous membranes of the mouth for the sole or prevalent purpose of cleaning them, perfuming them, modifying their appearance, protecting them, keeping them in good condition or correcting body odours”.

To classify a product as a cosmetic product, therefore, it is essential to consider:
the formulation,
the application site,
the function of the product.

Both the carriers and the inclusion of functional active ingredients in the formula which perform a biological but not pharmacological action contribute to the function of the product. These substances are the so-called “active ingredients”. These ingredients have a “claim” of effectiveness or there are studies to support the effect of these molecules on a biological process. For example, osmoprotectors belong to this category, i.e., those small organic molecules that, acting as osmolytes and concentrating themselves in the cytoplasm, allow cells to survive in conditions of osmotic stress, maintaining the osmotic balance between the intra and extracellular environment. They show a protective action on cellular proteins, promoting the osmotic balance and vitality of epidermal cells.

Cells of the same tissue communicate with each other through chemical signals sent from cell to cell to coordinate their activities. An ingredient with an osmoprotective claim reaches the deepest skin cells by exploiting the physiological transmission of cell-to-cell signals and triggering a repair process.

Cosmetic ingredients with hydrating action act in two different ways:
from outside: reducing trans-epidermal water loss. They are raw materials that form a barrier on the skin surface, for example film-forming agents and emollients, which prevent or reduce perspiration, retaining water inside and enhancing the skin barrier effect.
from inside: they contribute to increasing the water content inside the epidermis. They are therefore humectant and hygroscopic molecules (capable of absorbing and retaining water) thus helping to increase the amount of water present in the skin. The ingredients capable of exhibiting this action are also called real moisturizers.

The term “topical composition for cosmetic use” means, for the purposes of the present invention, various types of products such as: water-in-oil emulsions, oil-in-water emulsions, lotions, gels and oils for the skin, beauty masks, foundations (liquids, pastes, powders), face powder, talc for after-bath and body hygiene, beauty soaps, deodorant soaps, perfumes, toilet waters and cologne, preparations for baths and showers (salts, foams, oils, gel), hair removal products, deodorants and antiperspirants, hair dyes, waving, straightening and fixing products, styling products, hair cleaning products (lotions, powders, shampoos), products for keeping hair in shape (lotions, creams, oils), hair styling products (lotions, hairsprays, brilliantines), shaving products (creams, foams, lotions), make-up and make-up remover products, products intended to be applied to the lips, products for dental and mouth hygiene, nail care products and nail lacquers, products for external intimate hygiene, sun products, self-tanning products, products for lightening the skin and anti-wrinkle products.

To produce a plant extract, FU XII reports that the extracts are prepared by appropriate methods using ethanol or other suitable solvents. Suitable excipients can be added during the different stages of the production process, e.g. to improve the technological qualities of the product, such as homogeneity and consistency. Appropriate antimicrobial stabilizers and preservatives can also be added. (Capasso F. “Farmacognosia” chap.7, pag. 69-72; II ed. Springer).

Liquid extracts, according to FU XII page. 833, “are liquid preparations, in which, in general, one part by mass or volume is equivalent to one part by mass of the dried herbal drug or material of animal origin. These preparations are adjusted, if necessary, to meet the requirements for solvent content and, where appropriate, constituents.”

According to the definition of the Official Italian Pharmacopoeia, herbal drugs are essentially whole, fragmented or cut plants, plant parts, algae, fungi, lichens in an untreated state, generally in dried form, but sometimes fresh.

For the purposes of the present invention the wording “phytocomplex” consists of all those substances, pharmacologically active and not, that characterize the medicinal plant itself or the drug and thus represents its chemical composition, from which pharmacological and toxicological properties are derived (Capasso F. “FITOTERAPIA. Impiego razionale delle droghe vegetali” cap.20, page 249; Springer ed.) For the purposes of the present invention the term “eudermic extraction mixture” and/or “solvent mixture” and/or extraction mixture” means a mixture comparable to an extraction solvent in a process of preparing a liquid extract such as described above and comprising at least two cosmetic grade ingredients capable of acting as a solvent and extracting molecules/active ingredient from a herbal drug. At the same time, this “eudermic extraction mixture” acts as a carrier for the phytocomplex contained in it.

For the development of the present invention, the applicant started from the need to develop an extract starting from twigs and/or twigs of Morus alba L., containing a phytocomplex comprising two active ingredients such as Oxyresveratrol and Mulberroside A.

For the initial experiments the applicant used twigs and/or sprigs of Morus alba L. coming from prunings (waste material) of Gelsibachiculture plants located in northern Italy (Veneto region).

The liquid extract developed in the present invention includes two components called A and B. Component A is a phytocomplex extracted from twigs and/or sprigs of Morus alba L., comprising:
a) Oxyresveratrol
b) Mulberroside A Component B is a solvent mixture in which the aforementioned phytocomplex A. is dissolved and said solvent B. includes:
i. 1,3-propanediol
ii. Erythritol
iii. Water
in which:
I. the solvent mixture B. is the extraction solvent
II. the concentration of a. of the phytocomplex A is between 0.01 and 0.1% by weight of the total weight of the liquid extract, preferably between 0.02 and 0.05%
III. the concentration of b. of the phytocomplex A. is between 0.007 and 0.07% by weight of the total weight of the liquid extract, preferably between 0.01 and 0.04%
IV. the weight ratio (i.+ii.)/iii. of the solvent mixture B. is between 2.00 and 2.50, is preferably 2.33,
V. the weight ratio i./ii. of the solvent mixture B. is between 7.75 and 16.50, preferably between 10.66 and 13.00;
VI) weighted summation i.+ii. +iii. =100 Preferably the liquid extract described in the present invention is characterized by the fact that:
the water content iii. in the solvent mixture B. it is 30% by weight of the total weight of said solvent;
the concentration of 1,3-propanediol i. in the solvent mixture B. is between 62.5 and 64.5% by weight on the total weight of solvent B. and
the concentration of erythritol ii. in the solvent mixture B. is between 5.5 and 7.5% by weight of the total weight of said solvent.

The liquid extract according to the present invention can comprise an antioxidant preferably chosen between sodium metabisulfite and glutathione.

When the antioxidant is sodium metabisulfite the concentration of said antioxidant is preferably between 0.1 and 0.2% by weight on the total weight of solvent B.

When the antioxidant is glutathione, the concentration of said antioxidant is preferably between 0.005 and 0.1%, more preferably it is between 0.01 and 0.05% by weight on the total weight of solvent B.

The liquid extract is prepared from twigs and/or sprigs of Morus alba L. and the process of preparing the liquid extract includes the following stages:
1) Drying the twigs and/or sprigs of Morus alba L. at a temperature of 40° C. and up to a humidity lower than 10%
2) Grinding and sieving the dried product coming from stage 1 until obtaining a homogeneous powder with dimensions less than or equal to 6 mm
3) Preparing the solvent mixture B, including the following phases at room temperature:
3.1. mixing erythritol ii. in water iii. until complete dissolution,
3.2. addition of 1,3-propanediol under stirring until a clear solution is obtained;
4) Mixing of the dried powder obtained in stage 2. with the solvent mixture B. prepared in stage 3. in weight ratios dried powder/solvent between 1: 8 and 1: 12, preferably 1: 10 in an ultrasonic bath for 1 hour at room temperature:
5) Elimination of solid residues resulting from stage 4 through at least two vacuum filtrations using filters with a porosity of up to 0.22 microns.

The object of the present invention is a topical composition for cosmetic use comprising the liquid extract as described above, in which said liquid extract is preferably present in quantities between 1 and 4% by weight on the total weight of the cosmetic composition.

The liquid extract described in the present invention is used for the treatment and prevention of skin aging, in particular for treating and preventing the formation of skin blemishes.

The synergistic action of the components of the extract allows to obtain a topical skin composition with a global anti-aging action, i.e. a product that is able to prevent and treat skin aging by acting on different mechanisms involved in this process.

By way of an illustrative but not exhaustive list, these mechanisms are collagen degradation, production of free radicals, inflammation and inflammaging, mechanisms involved in cellular senescence, incorrect cell-cell communication due to senescence mechanisms, dysfunction of the skin barrier.

In view of the foregoing the cosmetic composition of the invention may be also interpreted as a cosmetic composition comprising:
Morus alba twigs and/or sprigs extract
propanediol i. and
erythritol ii..

In fact, the water present in the liquid extract of the invention cannot be distinguished from the water already present in the cosmetic composition when the latter is in the form of a water in oil or oil in water emulsion, for this reason the critical components present in the final cosmetic composition of the invention are Morus alba twigs and/or sprigs extracts, propanediol i. and erythritol ii.

Thus, except for the wt % contents, the Morus alba twigs and/or sprigs extract, propanediol and erythritol and the cosmetic composition are as described above, including advantageous and preferred embodiments. According to a particularly preferred embodiment of this cosmetic composition the content of propanediol i. is between 0.59% and 2.69%, preferably between 0.620 and 2.58%, more preferably between 0.625 and 2.58% by weight based on the weight of said cosmetic composition.

According to another preferred embodiment of this final composition erythritol ii. is comprised between 0.038 and 0.33%, preferably between 0.055 and 0.32%, more preferably between 0.055 and 0.3% by weight based on the weight of said cosmetic composition.

According to a further preferred embodiment, this cosmetic composition maintains the above mentioned the weight ratio i/ii of the liquid extract of the invention, that is comprised between 7.75 and 16.50, preferably between 10.66 and 13.00.

The Morus alba twigs and/or sprigs extract present in this cosmetic composition preferably comprise mulberroside A and oxyresveratrol.

Thus, except for the wt % contents, oxyresveratrol and mulberroside A are as described above, including advantageous and preferred embodiments. Preferably the oxyresveratrol is comprised between 0.0001% and 0.004%, more preferably from 0.0002% to 0.002% by weight based on the weight of said cosmetic composition.

Preferably the mulberroside A is comprised between 0.00007 and 0.0028%, preferably between 0,0001% and 0,0016% by weight based on the weight of said cosmetic composition.

These mulberroside A and oxyresveratrol content ranges above reported are easily derived from the conditions II)-III) of the liquid extract of the invention and also considering that the liquid extract is comprised in the cosmetic composition of the invention in amounts of from 1 to 4% by weight based on the total cosmetic composition weight.

The above compounds are preferably comprised in a phytocomplex extracted from sprigs and/or twigs of Morus alba. Said phytocomplex is as described above, including advantageous and preferred embodiments.

In case of the cosmetic composition in which the water content is lower than or equal to 0.05% by weight before the addition of the liquid extract of the invention, also the content of water present in the liquid extract, which is much higher, becomes a critical component in the final cosmetic composition of the invention containing the liquid extract of the invention.

Therefore, the water present in the cosmetic composition of the invention is preferably at least 0.28%, more preferably at least 0.30% by weight based on the total weight of the cosmetic composition.

According to a further preferred embodiment of the cosmetic composition of the invention the water content in wt % iii. is at least 0,4*(i.+ii.) preferably (i.+ii.)/2.33. The above formulas are easily derivable from the above mentioned condition IV) (i.+ii.)/iii of the liquid extract when it is equal to 2.5 or equal to 2.33.

All the range for propanediol, erythritol and water are easily derivable considering the abovementioned conditions of the liquid extract of the invention.

The composition of the invention may comprise an antioxidant, which is different from mulberroside A and oxyresveratrol. This antioxidant is as described above, including advantageous and preferred embodiments. It may be added notably to preserve mulberroside A and oxyresveratrol and is preferably selected from the above already mentioned, i.e. sodium metabisulfite and glutathione.

The above cosmetic composition is preferably in the form of topical compositions as described above. It thus presents the same use such as for the treatment and prevention of skin aging, in particular for treating and preventing the formation of skin blemishes. We report hereinbelow for illustrative but non limitative purposes the following examples.

### Examples

To identify the ingredients suitable for constituting the eudermic extraction mixture suitable for the purposes of the present invention, the applicant has developed carrying out various systematic extractions.

For each system, the applicant applied the following process:
1. Grinding the vegetable drug (twigs and/or sprigs of Morus alba L.)
2. Preparing the eudermic extraction mixture (solvent)
3. Extraction: the herbal drug was contacted with the eudermic extraction/solvent mixture in a ratio of 1:10 (w/w) and extracted in an ultrasonic bath for 1 hour at room temperature (25° C.).
4. Filtration of the extract: the exhausted plant drug is separated from the extraction solvent mixture by vacuum microfiltration with a Buchner funnel using filters with a porosity of up to 0.22 microns. This filtration method is commonly known to those skilled in the art.

The liquid extract object of the present invention corresponds to the liquid filtered in the last filtration step.

Steps 1 and 2 which are relevant for patenting purposes are described in detail below. Steps 3 and 4 are methods commonly known to those skilled in the art.

### Step 1—Preparation of the Herbal Drug for Extraction

The liquid extract of the present invention can be prepared starting from twigs and/or sprigs of Morus alba L.. The process of preparing the herbal drug involves a first step in which the twigs and/or sprigs are dried in an oven (air dryer) at a temperature of 40° C. until humidity is less than 10%.

The dried herbal drug is subsequently ground with a knife mill until obtaining a powder with a maximum grain size less than or equal to 6 mm, preferably less than or equal to 1 mm. This particle size is obtained by sieving the vegetal drug, dried and ground as previously described, with a sieve with a mesh between 6 mm and 1 mm which excludes powders larger than these dimensions.

Various extraction tests defined as “systematic extraction” were carried out on the herbal drug thus obtained, placing it in contact with different mixtures of ingredients. These mixtures are listed in table 2.

### Step 2—Preparation of the Eudermic Mixture as Extraction Solvent

The first systematic extraction served to investigate whether it was possible to combine two ingredients of normal cosmetic use in such a way as to produce a mixture capable of acting as an alternative solvent to a mixture of 70% ethanol in water. In fact, it is known from the literature that ethanol is a good solvent for extracting compounds such as Oxyresveratrol and Mulberroside A. from Morus alba L. plant material.

The ingredients shown in table 1 were also chosen assuming a synergy of action with the active ingredients extracted from the plant in order to produce an extract for cosmetic use with a “claim” global anti-aging effect.

The quantities used for each raw material are shown in table 2.

### Example 1: Identification of Carrier/Solvent Ingredients

The first systematic served to identify the ingredients that could compose the extraction mixture, each capable of satisfying the following characteristics:
Be a good carrier: i.e. ingredients with a “claim” of a functionality for cosmetic use. This step is particularly important because the applicant wanted to produce an extract in which the solvent mixture itself acted as a vehicle for the final extract. Compared to the prior art, therefore, after the extraction process no further steps are needed to eliminate/remove the solvent or purify the extract from any contaminants.
Be able to exert a solvent effect but at the same time be eudermic/suitable for skin application, non-irritating ingredients Produce a “ready to use” extract to be included in a formulation for cosmetic use. In this case we evaluate:
the physical state which must be liquid or powder soluble in water,
chemical-physical stability,
the colour,
viscosity;
the ability to protect the liquid extract from microbial contamination. This last aspect is very important because an aqueous extract is more exposed, compared to a dry extract, to chemical degradation processes of the extracted active ingredients and to microbial contamination.
cost of raw materials

The choice of the formulator/applicant was oriented towards raw materials containing at least one hydroxyl group, such as commercially available alcohols or polyols. 1,3-propanediol was chosen for its preservative function and its low viscosity, which facilitates filtration. The list of tested ingredients is shown in table 1.

TABLE 1
Ingredients chosen and used for extraction tests. For each raw material, the chemical
structure, physical form and cosmetic claim are also shown in the table.

| RAW MATERIAL | CHEMICAL STRUCTURE | PHYSICAL FORM | COSMETIC CLAIM |
|---|---|---|---|
| Sorbitol (70% in water) | 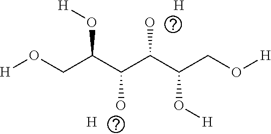 | Syrupy water miscible, clear, colorless liquid | Moisturizer |
| Erythritol | 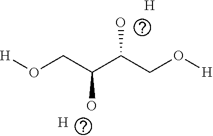 | White crystalline powder, easily soluble in water | Moisturizer, osmoprotectant |
| Trehalose | 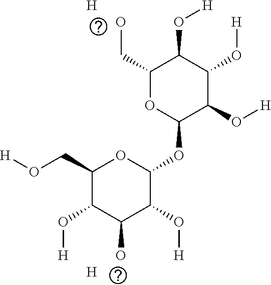 | White crystalline powder | Osmoprotectant, antioxidant |
| Xilitol | 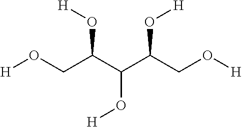 | White crystalline powder | Moisturizer |
| Myo-Inositol | 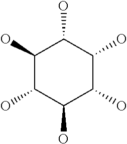 [SMILES: O[C@H]1[C@H](O)[C@@H](O)[C@H](O)[C@@H](O)[C@H]1O] | White crystalline powder | Moisturizer, osmoprotectant |
| Mannitol | 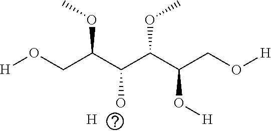 | White crystalline powder | Technological properties |
| 1,3-propanediol | 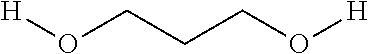 | Colorless liquid | Preservative function, good workability (low viscosity) |

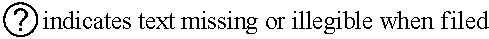

### Example 2: Extraction Tests with Different Mixtures of Ingredients in Table 1 in Order to Identify the Solvent Mixture Most Suitable for the Purposes of the Present Invention

For the extraction, the plant drug is mixed with the extraction mixture in a ratio of 1:10 (w/w) 5 and extracted in an ultrasonic bath for 1 hour at room temperature (25′Q).

Table 2 shows the extraction mixtures tested: for each extract, microbiological analysis and titration of the actives of interest (Oxyresveratrol and Mulberroside A) were performed.

For the microbiological analysis, an analysis test was performed following a standard protocol ISO 21148:2017, known to the skilled person in the field, and, in particular, ISO 21149:2017 for total bacterial load and ISO 16212:201 for yeasts and molds. For the cultured count of bacteria, yeasts and molds, the TSA and SDA media are used (TSA=Tryptic Soy Agar; SDA=Sabouraud Dextrose Agar). The microbial count values on TSA/SDA media are considered acceptable for a number <10 cfu/ml (cfu=colony forming units).

The titration of the actives was performed by HPLC-UV DAD. 1 g of biomass was weighed in 50 ml of methanol/water 80:20, sonicating for 40 minutes. The solutions were then centrifuged and placed in vials for analysis. The analyses were carried out in TPLC-DAD using a Phenomenex Synergy RP MAX column as the stationary phase and methanol and water with 0.30 formic acid as the mobile phases.

The stilbenoids oxyresveratrol and its glycosylated derivative mulberroside A (Sigma-Aldrich) were used as reference standards. The reading was conducted at 330 nm.

The T209 extract is the reference “gold standard” of 7000 ethanol extraction as disclosed in WO2020/201432.

TABLE 2
Extraction tests with microbiological analysis and titres of Oxyresveratrol and Mulberroside A

| Micorbiological analysis (cfu/ml) | | | | Stylben derivatives (% w/w) | | |
|---|---|---|---|---|---|---|
| Estratto | Composizione | TSA | SDA | Oxy | MulbA | Tot |
| T209 | Ethanol 70% | <10 | <10 | 0.027 | 0.033 | 0.06 |
| T204 | Sorbitol 10%, Trehalose10%, 1,3-propanediol 50%, | <10 | <10 | 0.011 | 0.029 | 0.04 |
| | water 30% | | | | | |
| T205 | Sorbitol 10%, Xylitol 10%, 1,3-propanediol 50%, | <10 | <10 | 0.012 | 0.03 | 0.042 |
| | water 30% | | | | | |
| T206 | Sorbitol 15.5%, Inositol 4,5%, 1,3-propanediol 50%, | <10 | <10 | 0.011 | 0.029 | 0.04 |
| | Water 30% | | | | | |
| T207 | Sorbitol 10%, Erythritol 10%, 1,3-propanediol 50%, | <10 | <10 | 0.011 | 0.031 | 0.042 |
| | Water 30% | | | | | |
| T208 | Sorbitol 13%, Mannitol 7%, 1,3-propanediol 50%, | <10 | <10 | 0.015 | 0.031 | 0.046 |
| | Water 30% | | | | | |
| T210 | Sorbitol 5%, Erythritol 5%, 1,3-propanediol 60%, | <10 | <10 | 0.018 | 0.033 | 0.051 |
| | Water 30% | | | | | |
| T211 | Sorbitolo 5%, Xylitol 5%, 1,3-propanediolo 60%, | 20 | <10 | 0.018 | 0.031 | 0.05 |
| | Water 30% | | | | | |
| T212 | Sorbitol 5%, Trehalose 5%, 1,3-propanediol 60%, | <10 | <10 | 0.018 | 0.033 | 0.051 |
| | Water 30% | | | | | |
| T213 | 1,3-propanediol 70%, Water 30% | <10 | <10 | 0.019 | 0.034 | 0.053 |
| T214 | Sorbitol 10%, 1,3-propanediol 60%, Water 30% | <10 | <10 | 0.018 | 0.034 | 0.053 |
| T215 | Xylitol 10%, 1,3-propanediol 60%, Water 30% | <10 | <10 | 0.018 | 0.036 | 0.054 |
| T244 | Erythritol 8%, 1,3-propanediol 62%, Water 30% | <10 | <10 | 0.034 | 0.038 | 0.072 |
| T290 | Erythritol 6%, 1,3-propanediol 64%, Water 30% | <10 | <10 | 0.039 | 0.021 | 0.06 |

Completely unexpectedly, the applicant found that the sum of the titres of the two active ingredients in the T244-T290 extracts are higher or comparable compared to the ethanol extraction.

The T244 and 1290 prototypes can extract both actives well. From this systematic extraction, the most promising prototypes from the point of view of the titre of the two active ingredients of interest were T244 and T290.

This systematic extraction was also used to evaluate the stability of the prototypes at T=5° C. and at room T (25° C.). This stability was performed over a period of six months. (Table 3)

TABLE 3
Thermal stability control

| | | refrigerator stability | room T stability | |
|---|---|---|---|---|
| Extract | Composition | results (5° C.)* | results | Extraction selection |
| T209 | Ethanol 70% | ok | ok | Control |
| T204 | Sorbitol 10%, Trehalose10%, 1,3- | ok | ok | Titre lower than |
| | propanediol 50%, Water 30% | | | control |
| T205 | Sorbitol 10%, Xylitol 10%, 1,3- | ok | ok | Titre lower than |
| | propanediol 50%, Water 30% | | | control |
| T206 | Sorbitol 15,5%, Inositol 4,5%, 1,3- | ok | ok | Titre lower than |
| | propanediol 50%, Water 30% | | | control |
| T207 | Sorbitol 10%, Erythritol 10%, 1,3- | ok | ok | Titre lower than |
| | propanediol 50%, Water 30% | | | control |
| T208 | Sorbitol 13%, Mannitol 7%, 1,3- | ok | ok | Titre lower than |
| | propanediol 50%, Water 30% | | | control |
| T210 | Sorbitol 5%, Erythritol 5%, 1,3- | ok | ok | Titre lower than |
| | propanediolo 60%, Water 30% | | | control |
| T211 | Sorbitolo 5%, Xilitolo 5%, 1,3- | ok | ok | Titre lower than |
| | propanediolo 60%, Acqua 30% | | | control |
| T212 | Sorbitolo 5%, Trealosio5%, 1,3- | ok | ok | Titre lower than |
| | propanediolo 60%, Acqua 30% | | | control |
| T213 | 1,3-propanediol 70%, Water 30% | ok | ok | Titre lower than |
| | | | | control |
| T214 | Sorbitol 10%, 1,3-propanediol 60%, | ok | ok | Titre lower than |
| | Water 30% | | | control |
| T215 | Xylitol 10%, 1,3-propanediol 60%, | ok | ok | Titre lower than |
| | Water 30% | | | control |
| T244 | Erythritol 8%, 1,3-propanediolo | ok | ok | Titre complying to the |
| | 62%, Water 30% | | | selected control |
| T290 | Erythritol 6%, 1,3-propanediolo | ok | ok | Titre complying to the |
| | 64%, Water 30% | | | selected control |

From these systematic experiments, the T244-T290 prototypes were selected as they demonstrated good solvent power (intended as a higher concentration of the actives of interest) and good stability at the temperature conditions tested.

Table 4 shows the prototypes selected to continue with the subsequent systematics.

TABLE 4
Names of the prototypes used in subsequent systematics and their
composition in % of the total weight of the liquid extract

| Prototype Number | Composition |
|---|---|
| T244 | Erythritol 8%, 1,3-propanediol 62%, |
| | water 30% |
| T290 | Water 6%, 1,3-propanediol 64%, water |
| | 30% |

### Example 3: Organoleptic Stability Check after One Month of the Selected Prototypes

To evaluate the organoleptic stability of the selected extracts after one month, a systematic study was set up in which each extract was placed under stress conditions, namely:
Fridge (5° C.)/dark
Room temperature (25° C.)/light
Oven (40° C.)/dark

In particular, the colour and visual appearance were analysed for each prototype: a darker colour variation, the presence of any precipitates or cloudiness of the solution indicate system instability.

The results are reported in table 5.

TABLE 5
Control of organoleptic stability at one month of the prototypes selected
in example 2. “OK” means no variation compared to T0 in terms
of colour and presence of precipitate or cloudiness of the sample.

| Prototype | | | Control times | |
|---|---|---|---|---|
| Number | solvent | Conditions | 1 week | 4 weeks |
| T244 | Erythritol 8%, 1,3- | Fridge Stabilit (5° C.)/ | ok | ok |
| | propanediolo 62%, | Dark | | |
| | Acqua 30% | Room Temperature/ | ok | It darkens |
| | | light | | slightly |
| | | oven (40° C.)/dark | ok | It darkens |
| | | | | slightly |
| T290 | Erythritol 6%, 1,3- | Fridge Stability (5° C.)/ | ok | ok |
| | propanediol 64%, | dark | | |
| | Water 30% | Rooom | ok | It darkens |
| | | temeperature/light | | slightly |
| | | oven(40° C.)/dark | ok | It darkens |
| | | | | slightly |

None of the tested extracts showed precipitation or cloudiness at four weeks, under all conditions tested. Both extracts showed a slight darkening in the light and in the oven while they showed no colour change at 5° C. in the dark.

### Example 4: Stability Tests with the Addition of an Antioxidant Agent

On the basis of the stability data of the T244 and T290 extracts, four different antioxidant systems were studied to preserve the active ingredients Oxyresveratrol and Mulberroside A which make up the phytocomplex and to prevent the browning of the liquid extract in the conditions tested in table 5 and for which a color change was detected.

Two antioxidant agents were chosen: sodium metabisulfite and glutathione.

Table 6 shows the prototypes studied and the conditions studied.

TABLE 6
Connection between names of prototypes studied in example
4 and related compositions including an antioxidant

| Prototype | |
|---|---|
| Number | Solvent Mixture |
| T244 | Erythritol 8%, 1,3-propanediol 62%, Water 30%, |
| T246 | Erythritol 8%, 1,3-propanediol 61.85%, Water 30%, |
| | Sodium metabisulfite 0.15% |
| T256 | Eritritolo 8%, 1,3-propanediol 61.9%, Water 30%, |
| | glutathione 0.1% |
| T257 | Erythritol 8%, 1,3-propanediolo 61.95%, Water 30%, |
| | glutathione 0.05% |
| T258 | Erythritol 8%, 1,3-propanediol 61.99%, Water 30%, |
| | glutathione 0.01% |
| T290 | Erythritol 6%, 1,3-propanediol 64%, Water 30% |
| T292 | Erythritol 6%, 1,3-propanediolo 63.85%, Water 30%, |
| | Sodium metabilsulfite 0.15% |
| T293 | Erythritol 6%, 1,3-propanediol 63.9%, Water 30%, |
| | glutathione 0.1% |
| T294 | Erythritol 6%, 1,3-propanediolo 63.95%, Water 30%, |
| | glutathione 0.05% |
| T295 | Erythritol 6%, 1,3-propanediol 63.99%, Water 30%, |
| | glutathione 0.01% |

TABLE 7
Control of the organoleptic stability at one month of the prototypes also including
an antioxidant chosen between sodium metabisulfite and glutathione.

| | | | Control times | |
|---|---|---|---|---|
| nr. prototype | Solvent | Conditions | T0 | 4 weeks |
| T292 | Erythritol 6%, 1,3- | Room temperature/ | | Ok color and it does |
| | propanediol 63.85%, | dark | | not precipitate |
| | Water 30%, | Fridge Stability (5° C.)/ | | Ok color and it does |
| | Sodium | dark | | not precipitate |
| | metabilsulfite | Oven (40° C.)/dark | | Ok color and it does |
| | 0.15% | | | not precipitate. |
| | | Micro TSA/SDA | <10 cfu/ml | <10 cfu/ml |
| T293 | Eyitritol 6%, 1,3- | Room temperature/ | | It darkens slowly and it |
| | propanediol 63.85%, | dark | | does not precipitate. |
| | water 30%, | Fridge Stability (5° C.)/ | | Ok color and it does |
| | Glutathione 0.1% | dark | | not precipitate. |
| | | Oven(40° C.)/dark | | It darkens slowly, and |
| | | | | it does not precipitate. |
| | | Micro TSA/SDA | <10 cfu/ml | <10 cfu/ml |
| T294 | Erythritol 6%, 1,3- | Room temperature/ | | It darkens slowly and it |
| | propanediol 63.85%, | buio | | does not precipitate. |
| | Water 30%, | Stabilita Frigo (5° C.)/ | | Ok color and it does |
| | Glutathione | dark | | not precipitate |
| | 0.05% | Stufa (40° C.)/buio | | It darkens slowly and it |
| | | | | does not precipitate. |
| | | Micro TSA/SDA | <10 cfu/ml | <10 cfu/ml |
| T295 | Erythritol 6%, 1,3- | Room temperature/ | | It darkens slowly, and |
| | propanediol 63.85%, | dark | | it does not precipitate. |
| | Water 30%, | Fridge Stability (5° C.)/ | | Ok color and it does |
| | Glutathione | dark | | not precipitate |
| | 0.01% | Oven (40° C.)/dark | | It darkens slowly and it |
| | | | | does not precipitate. |
| | | Micro TSA/SDA | <10 cfu/ml | <10 cfu/ml |

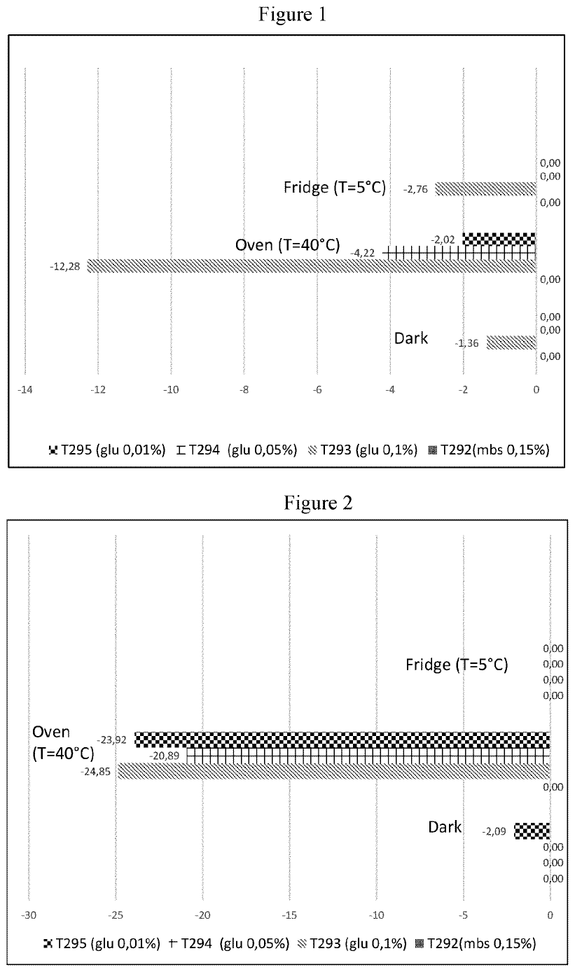

FIGS. 1 and 2 show the histograms relating to the decrease in Oxyresveratrol (FIG. 1) and Mulberroside A (FIG. 2) of the T292, T293, T294 and T295 extracts one month after preparation and subjected to the three conditions described in example 4, table 7. The values shown in the graph refer to the drop in 00 compared to the initial titre of active ingredient of each prototype.

The advantage of having used a mixture of ingredients commonly known in cosmetic formulations as a solvent lies in having obtained an extract which does not require further purification steps to eliminate the extraction solvent, because the extraction mixture is an eudermic mixture and concurs itself to the functionality and microbiological stability of the extract itself. To the knowledge of the applicant, there is no formula that is ready for use and stable, from a microbiological and organoleptic point of view, containing a phytocomplex including Oxyresveratrol and Mulberroside A.

The extract thus obtained can be used in cosmetic formulations for use in the prevention and treatment of physiological mechanisms linked to aging and for the well-being of the skin.

Below are some examples of formulations for illustrative but not limitative purposes.

### Face Lotion:

| Componente | range [% p/p] |
|---|---|
| Aqua | q.s. to 100 |
| Moisturizers | 1-20 |
| Rheological modifiers | 1-5 |
| Emulsifiers | 0.5-3 |
| Preservatives | 0.1-1.5 |
| Parfume | 0.1-1 |
| Hyaluronic acid | 0.01-1 |
| Liquid extract according to claim 1 | 1-4 |

| Componente | range [% p/p] |
|---|---|
| Aqua | q.s. to 100 |
| (Vegetable or mineral) oils waxes, fat (for | 1-5 |
| example long chain alcohols) | |
| Silicones | 1-10 |
| Moisturizers | 1-20 |
| Rheological modiifiers | 1-5 |
| Emulsifiers | 0.5-3 |
| Preservatives | 0.1-1.5 |
| Parfume | 0.1-1 |
| Hyaluronic acid | 0.05-1 |
| Liquid extract according to claim 1 | 1-4 |

### Solar Emulsion:

| Componente | range [% p/p] |
|---|---|
| Aqua | q.s. to 100 |
| (Vegetable or mineral) oils waxes, fat (for | 5-10 |
| example long chain alcohols) | |
| UV filters | 20-25 |
| Silicones | 1-10 |
| Moisturizers | 1-20 |
| Rheological modifiers | 1-5 |
| Emulsifiers | 0.5-3 |
| Presservatives | 0.1-1.5 |
| Parfumes | 0.1-1 |
| Chelating agents | 0.1-0.3 |
| Antioxidants | 0.03-0.5 |
| Hyaluronic acid | 0.01-1 |
| Liquid extract according to claim 1 | 1-4 |

1. Liquid extract comprising:
A. a phytocomplex extracted from sprigs and/or twigs of Morus alba L., comprising
a. Oxyresveratrol,
b. Mulberroside A
B. a solvent mixture in which said phytocomplex A is dissolved, said mixture comprising:
i. 1,3-propanediol
ii. Erythritol
iii. Water
wherein:
I. the mixture solvent B) is the extraction solvent
II. the concentration of a. of the phytocomplex A is comprised between 0.01 and 0.1% by weight on the total weight of the liquid extract, preferably between 0.02 and 0.05%
III. the concentration of b. of the phytocomplex A. is comprised between 0.007 and 0.07% by weight on the total weight of the liquid extract, preferably between 0.01 and 0.04%
IV. the weight ratio (i.+ii.)/iii. in the solvent mixture B. is comprised between 2.00 and 2.50, preferably is 2.33
V. the weight ratio i./ii. in the solvent mixture B. is comprised between 7.75 and 16.50, preferably it is comprised between 10.66 and 13.00
VI. the weight summation of i.+ii.+iii.=100

2. Liquid extract according to claim 1, wherein:
the content of water iii. in the solvent mixture B. is 30% by weight on the total weight of said solvent;
the concentration of 1,3-propanediol i. in the solvent mixture B. is comprised between 62.5 and 64.5% by weight on the total weight of the solvent B. and
the concentration of erythritol ii. in the solvent mixture B is comprised between 5.5 and 7.5% by weight of the total weight of said solvent.

3. Liquid extract according to claim 2, wherein the solvent mixture B. comprises an antioxidant.

4. Liquid extract according to claim 3, wherein said antioxidant is selected from sodium metabisulfite and glutathione.

5. Liquid extract according to claim 4, wherein when the antioxidant is sodium metabisulfite the concentration of said antioxidant is comprised between 0.1 and 0.2% by weight on the total weight of said solvent mixture B.

6. Liquid extract according to claim 4, wherein when the antioxidant is glutathione, the concentration of said antioxidant is comprised between 0.005 and 0.1%, preferably it is between 0.01 and 0.05% by weight on the total weight of said solvent mixture B.

7. Process for preparing the liquid extract according to claim 1 comprising the following steps:
1. drying sprigs and/or twigs of Morus alba L. at a temperature equal to 40° C. and up to a humidity of less than 10%;
2. grinding and sifting of the dried product from step 1 until a homogeneous powder with sizes less than or equal to 6 mm is obtained;
3. preparing the solvent mixture B., comprising the following steps at room temperature:
3.1. mixing of erythritol ii. in water iii. until complete dissolution,
3.2. adding 1,3-propanediol under stirring until a clear solution is obtained;
4. mixing the dried powder obtained in step 2. with the solvent mixture B. prepared in step 3. in weight ratios of dried powder/solvent mixture B. between 1: 8 and 1: 12 preferably 1: 10 in ultrasonic bath for 1 hour at room temperature; and
5. eliminating solid residues resulting from step 4 by at least two vacuum filtrations using filters with porosity up to 0.22 microns.

8. Cosmetic topical composition comprising the liquid extract according to claim 1.

9. Cosmetic topical composition according to claim 8, wherein said liquid extract is present in amounts comprised between 1 and 4% by weight on the total weight of the composition.

10. (canceled)

11. (canceled)

12. (canceled)

13. Cosmetic composition comprising:
Morus alba twigs and/or sprigs extract;
Propanediol i; and
erythritol ii.

14. Cosmetic composition according to claim 13, wherein the content of propanediol i. is between 0.59% and 2,69%, preferably between 0.620 and 2.58%, more preferably between 0.625 and 2.58% by weight based on the weight of said cosmetic composition.

15. Cosmetic composition according to claim 13, wherein the content of erythritol (ii) is between 0.038 and 0.33%, preferably between 0.055 and 0.32%, more preferably between 0.055 and 0.3% by weight based on the weight of said cosmetic composition.

16. Cosmetic composition according to claim 13, wherein the weight ratio (i)/(ii) is comprised between 7.75 and 16.50, preferably between 10.66 and 13.00.

17. Cosmetic composition according to claim 13, wherein said twigs and/or sprigs extract comprises mulberroside A and oxyresveratrol.

18. Cosmetic composition according to claim 17, wherein said oxyresveratrol is comprised between 0.0001% and 0.004% preferably from 0.0002% to 0.002% by weight based on the weight of said cosmetic composition.

19. Cosmetic composition according to claim 17, wherein said mulberroside A is comprised between 0.00007 and 0.0028%, preferably between 0,0001% and 0.0016% by weight based on the weight of said cosmetic composition.

20. Cosmetic composition according to claim 17, wherein said mulberroside A and said oxyresveratrol are comprised in a phytocomplex extracted from sprigs and/or twigs of Morus alba.

21. Cosmetic composition according to claim 13, containing water (iii) and optionally an antioxidant.

22. Cosmetic composition according to claim 21, wherein water is of at least 0.28%, preferably at least 0.30% by weight based on the total weight of the cosmetic composition.

23. Cosmetic composition according to claim 21 Or 22, wherein the water content is at least 0,4*[(i)+(ii)] preferably [(i)+(ii)]/2.33 in weight % based on the total weight of the composition.

24. Cosmetic composition according to claim 13, being a cosmetic topical composition.

25. A method for treating skin aging comprising administering topically to a subject in need thereof the topical composition according to claim 8.

26. A method for treating skin spots comprising administering topically to a subject in need thereof the topical composition according to claim 8.
